(19) United States
(12) Patent Application Publication
(10) Pub. No.: US 2008/0097237 A1
(43) Pub. Date: Apr. 24, 2008

(54) Method and system for detection of a cerebrovascular accident

(76) Inventors: Henry K. Gabaldon, Belen, NM (US); Esther I. Ferguson, Albuquerque, NM (US)

Correspondence Address: Intel Corporation;c/o DARBY & DARBY P.C., P.O. BOX 770, CHURCH STREET STATION, NEW YORK, NY, 10008-0770, US

(21) Appl. No.: 11/529,363

(22) Filed: Sep. 29, 2006

(51) Int. Cl. A61B 5/00

(52) U.S. Cl. 600/561

(57) ABSTRACT

An embodiment of the invention relates to a wireless attachment device, having an attachment section configured for attachment to a patient; an audio device configured to pick up pressure waves from the patient's brain and output data represention of the pressure waves; and a wireless transmitter connected to the audio device and configured to transmit the data representating the pressure waves. Another embodiment of the invention relates to a method for detecting a cerebrovascular accident comprises analyzing data representing pressure waves from blood vessels within a patient's head to recognize one or more signatures of a cerebrovascular accident. Yet additional embodiments relates to a system having the wireless attachment device.

### BACKGROUND OF THE INVENTION

1. Field of the Invention

This invention relates to the field of detecting and diagnosing a cerebrovascular accident or “stroke.” More specifically, this invention relates to detecting pressure waves in a patient's brain that are characteristic of stroke, and informing the patient or his caregiver of the impending or occurring stroke.

2. Background

Stroke is the leading cause of permanent disability in the U.S. and the third leading cause of death. Each year, 550,000 Americans have strokes. One-third of them die. Many of the survivors have decreased vocational function (71%); of these 16% remain institutionalized, and 31% need assisted care.

Stroke can result from vascular disease affecting the arteries supplying blood to the brain and occurs when one of these vessels bursts or is clogged, causing a part of the brain to be deprived of oxygen and nutrients that it needs to function. Nerve cells can die within minutes, and the part of the body controlled by these cells cannot function. The effects of a stroke can be permanent because the dead brain cells are not replaced.

There are two main types of strokes, ischemic and hemorrhagic. Clots—cerebral thrombosis or cerebral embolisms—cause ischemic strokes. Cerebral hemorrhage or subarachnoid hemorrhage causes hemorrhagic strokes. Ischemic strokes are the most common, but hemorrhagic strokes are the most deadly.

Cerebral thrombosis occurs when a blood clot (a thrombus) forms in an artery in or leading to the brain, blocking blood flow. It is the most common cause of ischemic stroke. Cerebral embolism occurs when a wandering clot (an embolus) or some other particle occurs in a blood vessel away from the brain, usually the heart. The clot is carried by the bloodstream until it lodges in an artery leading to or in the brain.

A cerebral hemorrhage occurs when an artery in the brain bursts, flooding the surrounding tissue with blood. Bleeding from an artery in the brain can be caused by a head injury or a burst aneurysm, a blood-filled pouch that balloons out from a weak spot in the artery wall. Hemorrhagic strokes cause loss of brain function both from loss of blood supply and from pressure of accumulated blood on surrounding brain tissue.

Diagnosing stroke occurrence, type, and severity takes time—time that stroke victims may not have. In addition, diagnosis occurs after the onset of stroke. Available diagnostic tools include tests that image the brain, such as computerized axial tomographic (CAT) scans, magnetic resonance imaging (MRI) scanning, and radionuclide angiography or nuclear brain scan. Tests that show the electrical activity of the brain are also used. The two basic tests of this type, an electro-encephalogram (EEG) and an evoked response test, measure how the brain handles different sensory stimuli such as flashes of light, bursts of sound, or electrical stimulation of nerves in an arm or leg.

Some types of stroke correlate to increased blood flow velocity, for example due to an occlusion in a blood vessel. A hemodynamic phenomenon that can occur as a result of increased blood flow velocity due to a sudden change in a blood vessel's diameter (from blood vessel occlusion) is the “water hammer effect.” The water hammer effect results from fluid impinging on the occluded area of the vessel and the hemodynamic energy that is expended in forcing the fluid through the occlusion, which causes rapid changes in pressure. It is therefore desirable to diagnose and perhaps prevent strokes caused by blood vessel occlusion by capturing, analyzing, and recognizing the pressure waves that are characteristic of the water hammer effect.

### BRIEF DESCRIPTION OF THE DRAWINGS

The accompanying drawings, which are incorporated herein and form part of the specification, illustrate the present invention and together with the description, further serve to explain the principles of the invention and to enable a person skilled in the pertinent art to make and use the invention. In the drawings, like reference numbers indicate identical or functionally similar elements.

FIG. 1 is a schematic diagram of a patient having wireless attachment devices attached to his head for stroke diagnosis and prediction in accordance with an embodiment of the invention.

FIG. 2 illustrates wireless attachment device used for stroke diagnosis and prediction in accordance with an embodiment of the invention.

FIG. 3 illustrates attachment of the wireless attachment device of FIG. 2 to a patient's skull.

FIG. 4 illustrates a long-period event.

### DETAILED DESCRIPTION OF THE INVENTION

As used in the specification and claims, the singular forms “a,” “an,” and “the” include plural references unless the context clearly dictates otherwise. For example, the term “an array” may include a plurality of arrays unless the context clearly dictates otherwise.

The present invention provides a method for detecting a cerebrovascular accident comprising analyzing data representing pressure waves from blood vessels within a patient's head to recognize one or more signatures of a cerebrovascular accident.

An embodiment of the invention relates to a wireless attachment device, comprising an attachment section configured for attachment to a patient; an audio device configured to pick up pressure waves from the patient's brain and output data representing the pressure waves; and a wireless transmitter connected to the audio device and configured to transmit the data representation of the pressure waves.

Preferably, the wireless attachment device comprises a bone screw and the attachment section comprises a shank with threads. Preferably, the audio device comprises an acoustic microphone. The wireless attachment device could further comprise an analog-to-digital converter. Preferably, the analog-to-digital converter is configured to receive analog data representing the pressure waves from the audio device and convert the analog data to digital data. Preferably, the wireless transmitter receives the digital data from the analog-to-digital converter. Preferably, the wireless attachment device comprises a bone screw, the audio device is provided at a distal end of a shank of the bone screw, the wireless transmitter is provided in a head of the bone screw, and a wire connecting the audio device to the wireless transmitter extends through the shank of the bone screw. Preferably, the attachment section comprises a pad configured to be adhesively bonded to the patient's head. Preferably, the wireless attachment device, the audio device and the wireless transmitter are integrated within the wireless attachment device to form a single device without any interconnecting electrical cables between the attachment device and the audio device or between the audio device and the wireless transmitter.

Another embodiment of the invention relates to a system for detecting a cerebrovascular accident, the system including a wireless attachment device and a wireless receiver remote from the wireless attachment device, the wireless attachment device comprising an attachment section configured for attachment to a patient; an audio device configured to pick up pressure waves from the patient's brain and output data representing the pressure waves; and a wireless transmitter connected to the audio device and configured to transmit the data representation of the pressure waves to the wireless receiver

In one embodiment, the invention is directed to a system for detecting a cerebrovascular accident, comprising a wireless attachment device for attachment to a patient's head, the wireless attachment device comprising an audio device and a wireless transmitter, a wireless receiver for receiving data wirelessly from the wireless transmitter, and a computer having a pattern recognition software thereon for analyzing the data received by the wireless receiver. The data includes pressure waves from blood vessels within the patient's head, and the pattern recognition software analyzes the data to recognize one or more signatures of a cerebrovascular accident.

The system could further comprise at least three of the wireless attachment devices for attachment to a patient's head. The system could further comprise a triangulation software, wherein the triangulation software is configured to determine a location of the cerebrovascular accident. Preferably, the wireless attachment device comprises a bone screw and the audio device is an acoustic microphone. Preferably, the bone screw is attached to the patient's skull and a tip of the acoustic microphone protrudes from a distal end of the bone screw when the bone screw is attached to the patient's skull.

In another embodiment, the invention is directed to a method for detecting a cerebrovascular accident, the method comprising attaching a wireless attachment device to a patient's head, the wireless attachment device comprising an audio device and a wireless transmitter, picking up pressure waves from blood vessels within the patient's head with the audio device, transmitting data representing the pressure waves, receiving the data representing the pressure waves, and analyzing the data received with a pattern recognition software to recognize one or more signatures of a cerebrovascular accident. The method could further comprise attaching at least three of the wireless attachment devices to the patient's head. Preferably, the analyzing the data further comprises recognizing three or more signatures of a cerebrovascular accident. The method could further comprise determining a location of the cerebrovascular accident. The method could further comprise converting analog input from the audio device to digital data for transmission by the wireless transmitter. Preferably, the attaching of the wireless attachment device to the patient's head includes attaching the wireless attachment device to the patient's skull. Preferably, the data is transmitted wirelessly.

Yet another embodiment of the invention relates to a method for detecting a cerebrovascular accident, the method comprising obtaining data representing pressure waves from blood vessels within a patient's head by the wireless attachment device, analyzing the data, and recognizing one or more signatures of a cerebrovascular accident. The method could further comprise triangulating a location of the cerebrovascular accident when three or more signatures of a cerebrovascular accident are recognized.

Further features of the present invention, as well as the structure of various embodiments of the present invention are described in detail below with reference to the accompanying drawings.

The present invention provides a device and method for identifying and predicting stroke events so that they can be prevented or at least diagnosed quickly and accurately. In an embodiment of the invention, one or more wireless attachment devices such as cranial pins or screws having integrated wireless communication capability, audio devices such as acoustic microphones, and analog-to-digital (A/D) converters are attached to a patient's head. The audio devices pick up pressure waves from the patient's brain, the A/D converters convert the pressure wave data from analog to digital, and a wireless transmitter sends the data wirelessly to a receiver. The wireless receiver then sends the data to be analyzed by a pattern recognition software that can identify a stroke event or predict an upcoming stroke event. The pattern recognition software can be run on, for example, a personal computer. If a stroke event is predicted or diagnosed, the patient or a caregiver is notified so that proper treatment can be obtained.

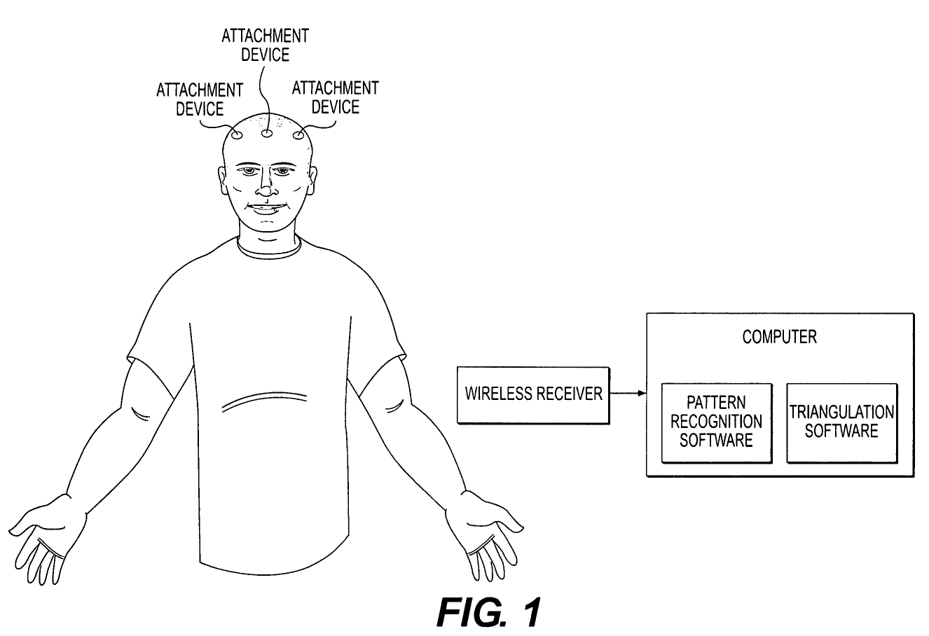

In another embodiment of the invention, as illustrated in FIG. 1, three or more wireless attachment devices such as cranial pins or screws having integrated Wi-Fi chips, microphones, and analog-to-digital (A/D) converters are attached to the patient's head in a known manner. The wireless attachment devices can be located on the two frontal and two occipital hemispheres of the skullcap. In order to triangulate one only needs two points to find a third point. For greater precision, three to four points are desirable. The cranial pins are to be placed as widely spread on the top region of the head as possible, whether it is three or more cranial pins. This is to maximize the coverage of the brain in order to pinpoint the CVA location by obtaining an X, Y, and Z coordinate, and can be used to triangulate the location of signature pressure waves, which indicate the location of the stroke event. Properly positioning the wireless attachment devices on the patient's head for triangulation can be accomplished using MRI or CAT scans, particularly in patients having incurred a head injury or patients in which an aneurism is suspected.

In a three screw formation, in order to optimize coverage of the skull, the screws are placed in a triangular formation as wide apart as possible on the top of the head. Likewise, in a four screw formation, in order to optimize coverage of the skull, the screws are placed in a rectangular formation as distally apart from each other as possible.

Triangulation is the process of finding coordinates and distance to a point by calculating the length of one side of a triangle, given measurements of angles and sides of the triangle formed by that point and two other known reference points. Determining coordinates and distance of the point can involve the solution of large meshes of triangles, with hundreds or even thousands of observations. Complex triangulation problems involving real-world observations with errors require the solution of large systems of simultaneous equations to generate solutions. The present invention contemplates using known triangulation software such as that used in MRI technology to locate the stroke event based on the data gathered and transmitted by the cranial pins. The triangulation software commonly utilizes Delaunay triangulation algorithms. The triangulation software may be run on, for example, an MRI machine or another computer such as a PC.

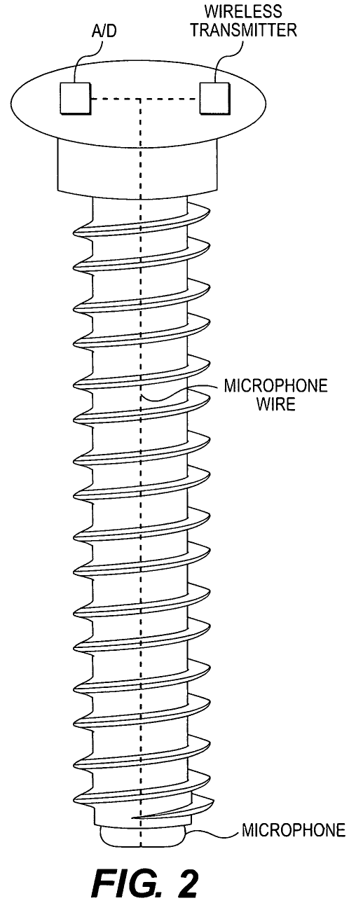

In an embodiment of the invention, as illustrated in FIG. 2, the wireless attachment device is a bone screw that comprises an attachment section illustrated as a screw shank with threads, and a head. As used herein, wireless attachment device generally means a device that is fixedly or removably attached. A wireless transmitter such as a Wi-Fi chip, an audio device such as an acoustic microphone, a microphone wire, and an A/D converter are housed within the bone screw. The wireless transmitter and the A/D converter are housed in the screw head, and the microphone is located at a distal portion of the shank (opposite the screw head) and is preferably arranged so that its tip protrudes from a distal end of the shank when the screw is attached to the patient's skull. The microphone wire extends through the shank to connect the microphone to the A/D converter. The A/D converter is connected to the wireless transmitter, which wirelessly communicates with a receiver. The present invention contemplates using a variety of types of suitable bone screws, which may comprise a variety of materials and have a variety of shapes and sizes.

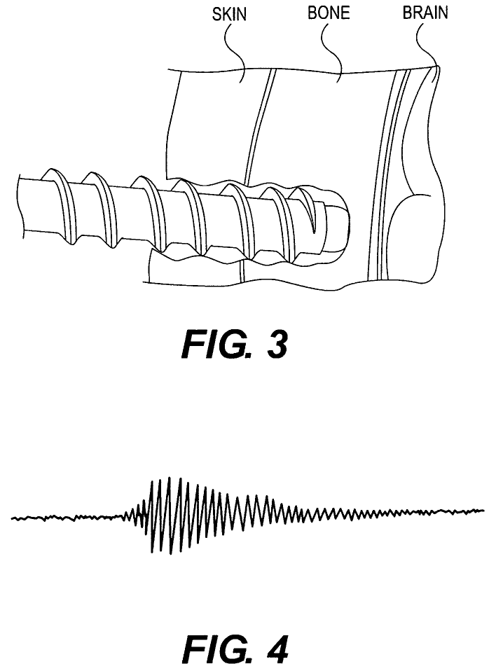

As illustrated in FIG. 3, in an embodiment of the invention utilizing bone screws, the bone screw is inserted through the patient's skin and into the underlying skull enough to anchor the screw; however, the screw does not extend through the skull to contact the patient's brain.

A microphone is an acoustic-to-electric transducer that converts sound or pressure waves into an electrical signal. In use, the microphone picks up sound or pressure waves from within the patient's brain and converts them to analog electronic signals. The analog electronic signals are sent via the microphone wire to the A/D converter, which converts the signals to digital data. The digital data is sent to the wireless transmitter for processing. The wireless transmitter sends the data to a wireless receiver.

Wi-Fi refers to an underlying technology of wireless local area networks based on the IEEE 802.11 specifications. Wi-Fi allows connectivity in peer-to-peer mode, which enables devices to connect directly with each other.

The invention detects and processes long-period events that are signatures of stroke by analyzing the pressure wave data detected and broadcast by the bone screws placed in the patient's head. The term “long period event” is used in the field of predicting volcanic eruptions, where long-period events typically refer to small pre-eruption earthquakes that emit lower frequency pressure waves. These lower frequency pressure waves from the pre-eruption earthquakes can be represented as shown in FIG. 4

The invention detects and processes long-period events created by sudden changes in pressure within blood vessels and arteries of the patient's brain. These long-period events reveal blood flow occlusions that increase blood flow velocity and create a water hammer effect and resulting rapid changes in pressure. The rapid changes in pressure are picked up by the microphone and converted to analog electronic signals. The signals are converted to digital data and the Wi-Fi chip then broadcasts the digital data to a receiver that sends the data to be processed by software that can recognize a stroke signature (long period event) and determine a location of the stroke signature to predict and/or diagnose stroke. The invention also contemplates informing the patient or a caregiver of impending or occurring stroke, along with a location of the recognized stroke signature, so that proper treatment can be given in a timely manner.

In an embodiment of the invention, the stroke signature within the broadcast pressure wave data is recognized by a pattern recognition software, such as Seisan Earthquake Analysis Software. The pattern recognition software recognizes the long-period event caused by pressure waves resulting from the water hammer effect within the patient's arteries, just as it would recognize a long-period event for earthquake analysis. Pattern recognition software differentiates long-period events that are stroke signatures from other wave patterns (noise). It then pulls the long-period event data from the pressure data that has been transmitted wirelessly by the bone screws in the patient's head. According to an embodiment of the invention, after detecting three long-period events that are determined to be signatures of stroke, the pattern recognition software sends the data to a triangulation software to pinpoint the location of the event. Thereafter, the patient or a caregiver can be notified of diagnosis or prediction of the location and occurrence of the stroke event.

### EXAMPLE

In an exemplary embodiment of the invention, three bone screws having an acoustic microphone, a microphone wire, an A/D converter, and a Wi-Fi chip are attached to a patient's head by screwing them into the skull a suitable distance to anchor them without penetrating through the skull. The bone screw is inserted into a patient's skull in any known and suitable method, which will be known to one skilled in the art The acoustic microphones in the bone screws pick up pressure waves from the blood vessels of the patients brain and send analog information representing the pressure waves via their respective microphone wires to respective A/D converters, which convert the analog information into digital data representing the pressure waves. The digital data is sent from the A/D converters to respective Wi-Fi chips, which transmit the digital data to a Wi-Fi receiver that is located suitably close to the patient to receive the wireless transmission.

The Wi-Fi receiver sends the data to be analyzed by Seisan Earthquake Analysis Software running on a PC. The Seisan software analyzes data representing the pressure waves to recognize one or more signatures of a cerebrovascular accident. If one or more signatures of a cerebrovascular accident are recognized by the software, a warning regarding an impending or occurring cerebrovascular accident is sent to the patient and/or a caregiver so that proper treatment can begin. In addition, if three or more signatures of a cerebrovascular accident are recognized by the software, information regarding the location of the signature with respect to each bone screw is sent to a triangulation software for triangulating a location of the cerbrovascular accident within the patient's head. The data sent is the wavelength and amplitude of the long period event wave the intersection of these wave points to the origin of the CVA. After a location is determined by the triangulation software, the patient and/or the caregiver is notified of the location of the cerebrovascular accident.

It is to be understood that the present invention contemplates other embodiments, including other wireless communications standards such as Bluetooth and Zigbee. Alternatively, Zigbee can be used in place of Wi-Fi. Zigbee refers to a specification for a suite of high-level communication protocols using small, low-power digital radios based on the IEEE 802.15.4 standard for wireless personal area networks (WPANs). ZigBee operates in the industrial, scientific and medical radio bands; 868 MHz in Europe, 915 MHz in the USA and 2.4 GHz in most jurisdictions worldwide. The technology is intended to be simpler and cheaper than other WPANs such as Bluetooth. In addition, the present invention contemplates using other devices than bone screws for attaching the Wi-Fi chip, A/D converter, and microphone to the patient's head. Indeed, a less permanent and invasive wireless attachment device can be used.

1. A wireless attachment device, comprising:
an attachment section configured for attachment within a patient's skull;
an audio device configured to pick up pressure waves from the patient's brain and output data representation of the pressure waves; and
a wireless transmitter connected to the audio device and configured to transmit the data representing the pressure waves, wherein the audio device is provided at a distal end of a shank of the bone screw.

2. The wireless attachment device of claim 1, wherein the attachment section comprises a shank with threads.

3. The wireless attachment device of claim 1, wherein the audio device comprises an acoustic microphone.

4. The wireless attachment device of claim 1, further comprising an analog-to-digital converter.

5. The wireless attachment device of claim 4, wherein the analog-to-digital converter is configured to receive analog data representing the pressure waves from the audio device and convert the analog data to digital data.

6. The wireless attachment device of claim 5, wherein the wireless transmitter receives the digital data from the analog-to-digital converter.

7. (canceled)

8. A system for detecting a cerebrovascular accident, the system including a wireless attachment device and a wireless receiver remote from the wireless attachment device, the wireless attachment device comprising a bone screw comprising:
an attachment section configured for attachment within a patient's skull;
an audio device configured to pick up pressure waves from the patient's brain and output data representing the pressure waves; and
a wireless transmitter connected to the audio device and configured to transmit the data representation of the pressure waves to the wireless receiver, wherein the audio device is provided at a distal end of a shank of the bone screw.

9. A method of manufacturing a wireless attachment device, the method comprising:
connecting an audio device to a wireless transmitter, the audio device being configured to pick up pressure waves from the patient's brain and output data representing the pressure waves, and the wireless transmitter being configured to receive transmit the data representation of the pressure waves; and
connecting the audio device and the wireless transmitter to an attachment section configured for attachment to a patient.

10. The method of claim 9, further comprising connecting an analog-to-digital converter between the audio device and the wireless transmitter, the analog-to-digital converter being configured to receive analog data representing pressure waves from the audio device and convert the analog data to digital data received by the wireless transmitter.

11. The system of claim 8, further comprising:
a computer having a pattern recognition software thereon for analyzing the data received by the wireless receiver,
wherein the data includes pressure waves from blood vessels within the patient's head, and the pattern recognition software is configured to analyze the data to recognize one or more signatures of a cerebrovascular accident.

12. The system of claim 11, comprising at least three of the wireless attachment devices for attachment to a patient's head.

13. The system of claim 12, further comprising a triangulation software, wherein the triangulation software is configured to determine a location of the cerebrovascular accident.

14. (canceled)

15. The system of claim 11, wherein the audio device is an acoustic microphone.

16. The system of claim 15, wherein the wireless attachment device further comprises an analog-to-digital converter for converting analog information from the acoustic microphone into digital data that is sent to the wireless transmitter for wireless transmission.

17. (canceled)

18. (canceled)

19. A method for detecting a cerebrovascular accident, the method comprising: attaching a wireless attachment device to a patient's head, the wireless attachment device comprising an audio device and a wireless transmitter;
picking up pressure waves from blood vessels within the patient's head with the audio device;
transmitting data representing the pressure waves;
receiving the data representing the pressure waves; and
analyzing the data received with a pattern recognition software to recognize one or more signatures of a cerebrovascular accident.

20. The method of claim 19, comprising attaching at least three of the wireless attachment devices to the patient's head.

21. The method of claim 20, wherein the analyzing the data further comprises recognizing three or more signatures of a cerebrovascular accident.

22. The method of claim 21, further comprising determining a location of the cerebrovascular accident.

23. The method of claim 19, further comprising converting analog input from the audio device to digital data for transmission by the wireless transmitter.

24. The method of claim 19, wherein the attaching the wireless attachment device to the patient's head includes attaching the wireless attachment device to the patient's skull.

25. The method of claim 19, wherein the data is transmitted wirelessly.

26. A method for detecting a cerebrovascular accident, the method comprising obtaining data representing pressure waves from blood vessels within a patient's head by the wireless attachment device of claim 1, analyzing the data, and recognizing one or more signatures of a cerebrovascular accident.

27. The method of claim 16 further comprising triangulating a location of the cerebrovascular accident when three or more signatures of a cerebrovascular accident are recognized.

28. The wireless attachment device of claim 1, wherein the attachment section comprises a pad configured to be adhesively bonded to the patient's head.

29. The wireless attachment device of claim 1, wherein the wireless attachment device, the audio device and the wireless transmitter are integrated within the wireless attachment device to form a single device without any interconnecting electrical cables located externally to the attachment device between the attachment device and the audio device or between the audio device and the wireless transmitter.

30. The system of claim 8 wherein the shank comprises threads.

31. The system of claim 8, wherein the attachment device comprises a pad configured to be adhesively bonded to the patient's head.

32. The system of claim 8, wherein the wireless attachment device, the audio device and the wireless transmitter are integrated within the wireless attachment device to form a single device without any interconnecting electrical cables located externally to the attachment device between the attachment device and the audio device or between the audio device and the wireless transmitter.

33. The wireless device of claim 1, wherein the wireless transmitter is provided in a head of the bone screw, and a wire connecting the audio device to the wireless transmitter extends through the shank of the bone screw.

34. The system of claim 8, wherein the wireless transmitter is provided in a head of the bone screw, and a wire connecting the audio device to the wireless transmitter extends through the shank of the bone screw.

35. The wireless device of claim 1, wherein the shank comprises threads.
